(19) United States
(12) Patent Application Publication
(10) Pub. No.: US 2025/0155365 A1
(43) Pub. Date: May 15, 2025

(54) PROCESS FOR DETECTING AMINE-TYPE COMPOUNDS IN AIR

(71) Applicants: COMMISSARIAT A L'ENERGIE ATOMIQUE ET AUX ENERGIES ALTERNATIVES, PARIS (FR); CENTRE NATIONAL DE LA RECHERCHE SCIENTIFIQUE, PARIS (FR)

(72) Inventors: Thu-Hoa TRAN THI, MONTROUGE (FR); Trung Hieu NGUYEN, PARIS (FR); Guillaume LE CHEVALLIER, GIF SUR YVETTE (FR)

(73) Assignees: COMMISSARIAT A L'ENERGIE ATOMIQUE ET AUX ENERGIES ALTERNATIVES, PARIS (FR); CENTRE NATIONAL DE LA RECHERCHE SCIENTIFIQUE, PARIS (FR)

(21) Appl. No.: 18/932,989

(22) Filed: Oct. 31, 2024

(30) Foreign Application Priority Data
Nov. 9, 2023 (FR) FR2312243

(51) Int. Cl. G01N 21/31; G01N 33/00

(52) U.S. Cl. CPC G01N 21/31; G01N 33/0027

(57) ABSTRACT

A nanoporous sensor composed of a silicate sol-gel matrix enclosing a reagents composition, the reagents composition including a mixture of 4-(dimethylamino) cinnamaldehyde and polystyrenesulfonic acid. Also, the process for preparing the nanoporous sensor composed of the silicate sol-gel matrix enclosing the reagents composition, and the use thereof in a process for detecting at least one amine-type compound, the compound being chosen from hydrazine, ethanolamine, ammonia and morpholine.

### FIELD

The present invention relates to a novel process for detecting and optionally quantifying amine-type compounds, in particular hydrazine (N₂H₄), in air.

### BACKGROUND

Hydrazine is classified as a CMR substance (IARC 2B-UE 1B). In 2011, it was incorporated into the REACH regulation candidate list of substances of very high concern. In 2017, the European Union lowered its TLV (8-hour Occupational Exposure Limit value) by a factor of 10, imposing a new threshold of 10 ppb (0.013 mg/m³), applicable from Jan. 17, 2020 at the latest. In addition to human health concerns, hydrazine is also classified as very toxic to aquatic organisms (EU classification H400 and H410).

Due to its CMR nature, hydrazine is no longer an oxidizer for rockets, but is still used in many fields, notably as an organic synthesis intermediate in the pharmaceutical and chemical industries, or as a blowing agent for polymer foams, or as a reducer of metal salts, or even as a corrosion inhibitor in industrial boiler circuit water, for example.

Due to its toxicity, assessing the risks associated with environmental hydrazine releases is important, and requires the ability to detect and optionally quantify the presence of hydrazine in the environment.

To date, this risk assessment is severely limited by the metrology associated with this substance, in particular as regards direct in-situ measurement. Specifically, although relatively sensitive and specific analytical methods do exist (final reading by HPLC-UV), they cannot be readily performed, nor are they compatible with industrial practices. “Instantaneous response” analytical methods are neither sufficiently selective nor sufficiently precise and sensitive to allow expected hydrazine gas concentrations in final discharges to be evaluated.

Hydrazine detection and quantification is currently performed using an indirect method. This method involves collecting the ambient air to be analyzed and trapping the hydrazine in an acidified cartridge, then desorbing it and reacting it with a reagent solution. The adducts formed are then separated by liquid chromatography and analyzed optically.

In addition to the duration of this method, which involves several steps that are difficult to perform directly at the sampling site, potential interference with other gaseous nitrogen compounds is not known, notably in the potential presence of interferents used to basify water or resulting from the degradation of hydrazine, such as ethanolamine (NH₂EtOH), morpholine or ammonia (NH₃).It is important to be able to measure hydrazine selectively in a direct manner in a concentration range from 1 to 100 ppb in dry to very humid air and in the presence of interferents whose concentration may be 50 to 100 times higher than that of hydrazine.

The process which is a subject of the present invention provides a solution to these problems.

Specifically, the process according to the invention allows hydrazine to be detected and optionally quantified selectively in the presence of water vapor, volatile organic compounds and other nitrogen compounds such as ethanolamine (NH₂EtOH), morpholine or ammonia. On contact with hydrazine, the sensor used changes color and the color intensity is proportional to the hydrazine content, allowing this compound to be detected and potentially quantified.

### PRIOR ART

The detection of hydrazine has been the subject of numerous studies, and detection methods are as numerous as they are varied, due to the high chemical reactivity of this compound. As a result, only those methods proposed in the literature are described here, which are capable of covering the required concentration range from 1.3 to 130 μg·m⁻³ (i.e. 1 to 100 ppb).

Methods for measuring hydrazine in air are less numerous than those for the liquid phase, notably in the target range of 1.3 to 130 μg·m⁻³ (i.e. 1 to 100 ppb). The current measurement method is described in INRS Data Sheet 21. It is based on the use of benzaldehyde [¹]. Hydrazine is collected by air suction through a tube filled with an inert adsorbent (Chromosorb P NAW or equivalent) of 3060 Mesh particle size, impregnated with sulfuric acid. The cartridge contents are desorbed with deionized water and benzaldehyde derivatization is performed. The adduct, benzalazine, is measured by liquid chromatography (HPLC) coupled with UV optical detection [^{2, 3}]. This method allows 30 ppb of hydrazine to be detected within 15 minutes of sampling.

A variant of this method is the use of cassettes containing two glass fiber filters impregnated with sulfuric acid. The hydrazine extraction is performed with an EDTA buffer solution and the derivatization is performed with benzaldehyde. The benzalazine formed is measured by liquid chromatography coupled with UV optical detection [⁴]. This assay method proves to be sensitive, since it is possible to detect 0.017 ppb of N₂H₄.

These two methods do not allow direct measurement at the sampling site, and the interference of ammonia or other amines at high concentrations 50 to 100 times that of hydrazine is not known.

Direct measurement methods are also available. In the context of monitoring worker exposure to hydrazine, monomethylhydrazine (MMH) and 1,1-dimethylhydrazine (UDMH) used as rocket fuels at air bases and space centers in the USA, several colorimetric dosimeters have been developed and sold by laboratories [^{5,6}]. The principle is based on the incorporation of an aromatic aldehyde such as vanillin, para-dimethylaminobenzaldehyde (pDMAB) or 2,4-dinitrobenzaldehyde on filter paper or an inert surface. Hydrazine and MMH react with vanillin or 2,4-dinitrobenzaldehyde to form a yellow-colored compound, while the product formed with pDMAB is orange. UDMH reacts with 2,4-dinitrobenzaldehyde to form a yellow-colored compound, and thus there is no reaction with vanillin and pDMAB. Colorimetric dosimeters based on vanillin and para-dimethylaminobenzaldehyde are sold by the company DODTEC [⁷] and CHEMSEE [⁸]. They do not allow exact quantification, but only the estimation of hydrazine and monomethylhydrazine contents in air from 25 ppb up to 1.2 ppm.

In patent U.S. Ser. No. 00/571,9061 A [⁹], Rose-Pehrsson et al. propose a process allowing the selective detection and quantification of liquid or gaseous hydrazine, monomethylhydrazine and 1,1-dimethylhydrazine by derivatization with aromatic carboxyaldehydes and fluorimetric analysis. Selectivity is based on the reactivity of three derivatizing agents, ortho-phthalaldehyde (OPA), napthalene-2,3-dicarboxaldehyde (NDA) and anthracene-2,3-dicarboxaldehyde (ADA) with hydrazine, monomethylhydrazine and 1,1-dimethylhydrazine as a function of the pH of the reagent solution. These authors thus developed a complex device which allows ambient air to be pumped into and sparge a reagent solution whose composition (OPA or NDA or ADA) and pH must be modified to produce selectivity. Although the detection limit reached is ppb, analysis of the gas mixture to be analyzed requires numerous reagent and pH change steps, followed by fluorimetric analysis. Moreover, there was no study of interference, notably with other amines at concentrations 50 to 100 times higher, which are liable to modify the pH of the solution.

For precise measurements, a number of commercial devices are available. The portable electrochemical detector from the company Interscan, model 4180-100b [¹⁰] allows detection of hydrazine in the range 10 to 100 ppb in less than 1 second, with a high detection limit of 10 ppb. The method is also non-selective, as the sensor also detects NH₃, NOx, CO and other organic amines.

High sensitivity may be obtained using devices equipped with a photoionization detector (PID), such as ppbRAE 3000 from the company RAE [¹¹]. The photoionization detector, equipped with a 10.6 eV lamp, allows hydrazine ionization and measurement of a few ppb in 3 seconds. Detection is not selective, however, as a large number of volatile organic compounds present in the air with ionization potentials below 10.6 eV are also detected, such as NH₃, ethanolamine and morpholine.

Hydrazine ionization followed by ion mobility measurement using an Ion Mobility Spectrometer (IMS) allows contents of about ten ppb (20-30 ppb) to be reached, while at the same time being selective in terms of the choice of carrier gas. When using a radioactive source, the IMS (such as the SABRE 4000 portable detector [¹²] or Environics' ChemPro 100i [¹³]) must be under the responsibility of a person competent in radiation protection. This technique is favored by armies and police forces for the detection of chemical weapons and illicit products. Its application in the public domain has developed more recently with the development of new non-radioactive ionization sources (corona effect) such as the PAIMS portable detector from the company MaSaTECH [¹⁴] or LCD 3.3 from Smiths Detection [¹⁵].

The prior art in hydrazine measurement shows that the only simple method currently adopted, using benzaldehyde as the reagent, can be used with good selectivity and sensitivity. However, hydrazine measurement in the air compartment requires adsorption and then desorption and derivatization steps, followed by optical analysis, which are difficult to perform on site. Furthermore, for this method, interference with high concentrations of NH₃, ethanolamine or morpholine gas in the atmosphere is not known.

There is thus a real need for a hydrazine-selective process for the direct detection and, optionally, direct quantification of hydrazine in air, which is compatible with the presence of high concentrations of interferents and readily performable on site.

The process according to the invention addresses these issues.

### SUMMARY

A first subject of the invention is a nanoporous sensor composed of a nanoporous silicate sol-gel matrix enclosing a reagents composition, said reagents composition comprising a mixture of 4-(dimethylamino) cinnamaldehyde and polystyrenesulfonic acid.

Another subject of the invention is a process for preparing a nanoporous sensor according to the invention, said process comprising the following steps:
a. synthesis of a sol from an organosilyl precursor, the synthesis being performed in a solvent, said solvent comprising water, in the presence of 4-(dimethylamino) cinnamaldehyde and polystyrenesulfonic acid;
b. gelation of the sol obtained in step a), so as to obtain a gel;
c. drying of the gel obtained in step b) so as to obtain a nanoporous sensor.

A subject of the present invention is also a process for detecting at least one amine-type compound, said compound being chosen from hydrazine, ethanolamine, ammonia and morpholine, using a nanoporous sensor according to the invention, said process comprising the steps of:
a. placing a gas sample to be analyzed in contact with said nanoporous sensor.
b. detecting said amine-type compound(s) on said nanoporous sensor.

Another subject of the invention is the use of a nanoporous sensor according to the invention, for the detection and/or quantification of at least one amine-type compound, said compound being chosen from hydrazine, ethanolamine, ammonia and morpholine.

The present invention also relates to a device for detecting at least one amine-type compound, said compound being chosen from hydrazine, ethanolamine, ammonia and morpholine, in a gas sample to be analyzed, said device comprising a cell enclosing a nanoporous sensor according to the invention and comprising:
a gas inlet;
a gas outlet;
an optical input;
an optical output.

### BRIEF DESCRIPTION OF THE DRAWINGS

Other features, details and advantages will be seen on reading the detailed description hereinbelow, and on analysis of the appended drawings, on which:

FIG. 1 shows the scheme for the reaction between N₂H₄ and DMACA catalyzed in an acidic medium, with the formation of DMACA-N₂H₄ (compound 3) and 2DMACA-N₂H₄ (compound 4).

FIG. 2 shows the differential spectra obtained at different times showing the evolution of the absorbance of the DMACA-N₂H₄ and 2DMACA-N₂H₄ complexes in the Hy3 nanoporous sensor during exposure of the sensor to a stream of gas mixture containing 14 ppb of N₂H₄. A spectrum is collected every 5 min from 0 to 60 min.

FIG. 3 shows the differential spectra obtained at different times showing the evolution of the absorbance of neutral DMACA in the Hy1 nanoporous sensor during exposure of the sensor to a stream of gas mixture containing 5 ppm of NH₃. A spectrum is collected every 5 min from 0 to 50 min.

FIG. 4 shows a scheme of an example of a device according to the invention.

FIG. 5 shows a scheme of an example of gas stream flow around the nanoporous sensor placed in the cell according to a particular embodiment of the invention.

FIG. 6 shows a scheme of an example of a cell of the device according to the invention.

FIG. 7 shows: on the left, the evolution of the absorption spectrum of the Hy1 sensor exposed to 5 ppm of NH₃, a spectrum is collected every 5 minutes for 50 minutes; on the right, the variation in the rate of formation of neutral DMACA as a function of NH₃ concentration. Stream=200 mL·min⁻¹, % RH=50%.

FIG. 8 shows the calibration curve of the Hy3 sensor for the detection of N₂H₄. Variation of IDMACA-N₂H₄ complex formation rate at 388 nm as a function of N₂H₄ concentration. Stream=200 mL·min⁻¹, % RH=50%.

FIG. 9 shows the effect of the relative humidity of the gas mixture on the rate of formation of the 1DMACA-N₂H₄ complex as a function of N₂H₄ concentration. Stream=200 mL·min⁻¹.

FIG. 10 shows the effect of the presence of a potential interferent, NH₂EtOH, on the response of the Hy3 sensor to hydrazine. Stream=200 mL·min⁻¹, RH=50%.

FIG. 11 shows the effect of the presence of a potential interferent, morpholine, on the response of the Hy3 sensor to hydrazine. Stream=200 mL·min⁻¹, RH=50%.

FIG. 12 shows the effect of the presence of a potential interferent, NH₃, on the response of the Hy3 sensor to hydrazine. Stream=200 mL·min⁻¹, RH=50%.

FIG. 13 shows the effect of the presence of two potential interferents, NH₃ and NH₂EtOH, on the response of the Hy3 sensor to hydrazine. Stream=200 mL·min⁻¹, RH=50%.

FIG. 14 shows the effect of the presence of two potential interferents, NH₃ and morpholine, on the response of the Hy3 sensor to hydrazine. Stream=200 mL·min⁻¹, RH=50%.

FIG. 15 shows a comparison of Hy1, Hy2 and Hy3 sensor responses to 30 ppb of N₂H₄. Stream=200 mL·min⁻¹, % RH=50%.

FIG. 16 shows a comparison of the responses of Hy8 and Hy9 sensors to 40 ppb of N₂H₄. Stream=200 mL·min⁻¹, % RH=50%.

FIG. 17 shows a comparison of the responses of Hy4, Hy5, Hy6 and Hy7 sensors to 25 ppb of N₂H₄. Stream=200 mL·min⁻¹, % RH=50%.

### DETAILED DESCRIPTION

The subject of the present invention is a nanoporous sensor composed of a nanoporous silicate sol-gel matrix enclosing a reagents composition, said reagents composition comprising a mixture of 4-(dimethylamino) cinnamaldehyde and polystyrenesulfonic acid.

For the purposes of the present invention, silicate sol-gel matrix means a material obtained via a sol-gel process which consists in using silicon alkoxides of formula Si(OR)x, where R is an alkyl group, as precursors.

During the sol-gel process, alkoxy groups (OR) are hydrolyzed in the presence of water to form silanol groups (Si—OH). These condense to form siloxane bonds (Si—O—Si—). The result is small particles, generally less than 1 μm in size, which aggregate to form clusters that remain in suspension without precipitating, forming a sol. The increase in clusters and their condensation increase the viscosity of the medium, which then gels. A porous solid material is obtained by drying the gel, with the solvent being expelled from the polymeric network formed (syneresis). Sol-gel matrices obtained from silicon alkoxides of formula Si(OR)ₓ are referred to as silicate sol-gel matrices in the present patent application.

For the purposes of the present invention, the term “nanoporous” means having pores with a size of less than 100 nm.

According to a particular embodiment, the nanoporous matrix according to the invention is an essentially microporous matrix. Thus, according to this embodiment, the sensor may also be described as essentially microporous. An essentially microporous material (essentially microporous sensor or essentially microporous matrix) means a material in which at least 80% of the pores are micropores.

According to the IUPAC definition, micropores are characterized by a width not exceeding 2 nm (IUPAC. Compendium of Chemical Terminology, 2nd ed. (the “Gold Book”). Compiled by A. D. McNaught and A. Wilkinson. Blackwell Scientific Publications, Oxford (1997). Online version (2019-) created by S. J. Chalk. ISBN 0-9678550-9-8. https://doi.org/10.1351/goldbook).

On contact with an amine-type compound, notably gaseous hydrazine, the nanoporous sensor according to the invention changes color due to the formation of DMACA-N₂H₄ and 2DMACA-N₂H₄ between hydrazine (N₂H₄) and 4-(dimethylamino) cinnamaldehyde (DMACA) in the presence of polystyrenesulfonic acid, which allows this reaction to be catalyzed. The absorbance variations are correlated with hydrazine content in the air.

The reaction between hydrazine (N₂H₄) and 4-(dimethylamino) cinnamaldehyde (DMACA) is advantageously catalyzed by an acid derived from a polymer, in particular polystyrenesulfonic acid. The choice of this type of acid, and in particular polystyrenesulfonic acid, has many advantages.

Firstly, this acid is a high molecular weight polymer including an SO₃H acid function for each styrene monomer. As a result, the number of available protons is particularly high, allowing the pore acidity to be adjusted to catalyze the reaction between DMACA and N₂H₄.

Moreover, inorganic acids and organic acids of low molecular weight have the drawback that acid vapors may be released during the step of drying the sol-gel matrix, which may result in evaporation of the acid in this case too. Thus, a volatile inorganic acid such as HCl (or an organic acid such as acetic acid) could evaporate from the sol-gel matrix, resulting in a loss of efficacy, or even preventing the sensor from functioning. This drawback is not present for acids derived from a polymer, such as polystyrenesulfonic acid, as these acids are not volatile.

Finally, another major advantage of polymer-derived acids relates to the deployment of acidic polymer chains in the small pores of the sol-gel matrix. This deployment of acidic functional groups in the pores makes it possible, firstly, to acidify each pore and, secondly, to strongly restrict polymer diffusion into the pore network, preventing it from migrating toward the sensor surface.
The advantages of this method over that currently used with hydrazine trapping in cartridges followed by delayed derivatization with the benzaldehyde reagent are numerous. The sensor according to the invention allows: direct on-site hydrazine measurement;
hydrazine measurement, notably by measuring the absorbance of the DMACA-N₂H₄ adduct at 388 nm (FIG. 1);
hydrazine measurement by sampling the air to be analyzed at a flow rate that may range from 10 to 600 mL·min⁻¹, for example a flow rate of 200 mL·min⁻¹, the sampling time possibly varying as a function of the concentration of the gas to be detected, for example between 60 min and 5 min, here respectively for 1 and 100 ppb of detected hydrazine;
selective measurement of hydrazine in the 1-85 ppb concentration range in the presence of interferents such as NH₃, ethanolamine (NH₂EtOH) or morpholine, when the total concentration of potential interferents is less than 200 times the hydrazine concentration ([N₂H₄]);
selective measurement of hydrazine in gas mixtures with a relative humidity ranging between 25% and 100%.

Advantageously, the nanoporous sensor according to the invention is characterized by a large specific surface area for adsorption. Specifically, it has a specific surface area for adsorption of from 700 to 2500 m²·g⁻¹, preferably from 800 to 2000 m²·g⁻¹.

The specific surface area for adsorption, pore volume and pore size distribution are determined by analyzing the liquid nitrogen adsorption-desorption isotherm using the Density Functional Theory (DFT) model. The BET (Brunauer-Emmett-Teller) method is an analytical method that enables deduction of the specific surface area for adsorption.

The nanoporous sensor according to the invention preferentially has a pore volume of 0.1 to 0.9 cm³·g⁻¹, preferably of 0.2 to 0.8 cm³·g⁻¹, even more preferentially of 0.2 to 0.6 cm³·g⁻¹. The pore volume represents the volume occupied by the pores per gram of sensor. The pore volume of the material is obtained from the nitrogen adsorption isotherm at liquid nitrogen temperature.

Advantageously, the nanoporous sensor according to the invention has a proportion of micropores greater than 75%, preferably greater than 80%, more preferably ranging from 85% to 95%, the remainder to 100% corresponding to the proportion of mesopores.

According to the IUPAC definition (IUPAC. Compendium of Chemical Terminology, 2nd ed. (the “Gold Book”). Compiled by A. D. McNaught and A. Wilkinson. Blackwell Scientific Publications, Oxford (1997). Online version (2019-) created by S. J. Chalk. ISBN 0-9678550-9-8. https://doi.org/10.1351/goldbook), the term “micropores” means pores with a width not exceeding 2 nm, and “mesopores” means pores with a width of between 2 and 50 nm. Pores with a width greater than 50 nm are referred to as macropores according to the same IUPAC reference.

The nanoporous sensor according to the invention preferentially has a mesopore content of less than 25%, preferably less than 20%, and more preferentially ranging from 5% to 15%, the remainder to 100% corresponding to the micropore content.

In particular, the nanoporous sensor according to the invention may have micropores with a diameter ranging from 0.3 to 2 nm, preferably from 0.5 to 2 nm.

The nanoporous sensor according to the invention may also be characterized in that it advantageously has mesopores with a diameter of between 2 and 20 nm, preferably between 2 and 15 nm.

The nanoporous sensor according to the invention preferably has a ratio r of polystyrenesulfonic acid and 4-(dimethylamino) cinnamaldehyde concentrations of 1 to 20, preferably 2 to 15, even more preferentially 10.

This ratio is calculated from the molar concentrations of polystyrenesulfonic acid and 4-(dimethylamino) cinnamaldehyde (r=[H⁺]/[DMACA]). For this calculation, the molar mass of the polystyrenesulfonic acid monomer (M=184 g/mol) is used so as to convert the polystyrenesulfonic acid (PSS) mass concentration into a molar concentration. For example, a PSS concentration of 9.2 g·L⁻¹ corresponds to a concentration of 0.05 mol·L⁻¹ (=9.2/184). Thus, with a DMACA concentration of 5.10⁻³ mol·L⁻¹ and a PSS concentration of 9.2 g·L⁻¹ (0.05 mol·L⁻¹), the ratio r is equal to 10.

The ratio of the concentrations of polystyrenesulfonic acid and 4-(dimethylamino) cinnamaldehyde may also be expressed as a ratio r′ of the mass concentrations of the two species (r′=[H⁺]/[DMACA]), this ratio r′ ranging from 1 to 20, preferably from 2 to 15, even more preferentially 11. For example, with a DMACA concentration of 5.10⁻³ mol·L⁻¹ corresponding to 0.876 g·L⁻¹ (with M=175.23 g·mol⁻¹) and a PSS concentration of 9.2 g·L⁻¹, the ratio r′ is equal to 10.5.

Another subject of the present invention is the process for preparing the nanoporous sensor according to the invention, said process comprising the following steps:
a. synthesis of a sol from a chosen organosilyl precursor, the synthesis being performed in a solvent, said solvent comprising water, in the presence of 4-(dimethylamino) cinnamaldehyde and polystyrenesulfonic acid;
b. molding of the sol obtained in step a), followed by gelation to obtain a gel;
c. drying of the gel obtained in step b), followed by demolding to obtain a nanoporous sensor.

According to a preferred embodiment of the process according to the invention, the organosilyl precursor is chosen from precursors with rapid hydrolysis and condensation and no long hydrophobic chains, such as tetramethoxysilane Si(OCH₃)₄), methyltrimethoxysilane CH₃Si(OCH₃)₃, ethyltrimethoxysilane (C₂H₅)Si(OCH₃)₃, 3-aminopropyltriethoxysilane (C₃H₆NH₂)Si(OC₂H₅)₃, 3-aminopropyltrimethoxysilane (C₃H₆NH₂)Si(OCH₃)₃), (3-(methylamino)propyl)trimethoxysilane (C₃H₆NHCH₃)Si(OCH₃)₃, 3-carboxypropyltriethoxysilane (C₃H₆CO₂H)Si(OC₂H₅)₃, 3-carboxypropyltrimethoxysilane (Si(C₃H₆CO₂H) (OCH₃)₃), tetraethoxysilane and mixtures thereof. The precursor is preferentially chosen from tetramethoxysilane (TMOS), tetraethoxysilane (TEOS) and mixtures thereof.

Step a) of the process according to the invention is preferentially performed in a solvent which may be water or a water/alcohol mixture, the alcohol preferably being a C1 to C6 aliphatic alcohol, more preferentially methanol or ethanol, or a mixture of water with a solvent chosen from acetone, formamide and methyl ethyl ketone.

In the process according to the invention, the sol synthesis step a) is advantageously performed first by mixing an organosilyl precursor chosen from tetramethoxysilane Si(OCH₃)₄), methyltrimethoxysilane CH₃Si(OCH₃)₃, ethyltrimethoxysilane (C₂H₅)Si(OCH₃)₃, 3-aminopropyltriethoxysilane (C₃H₆NH₂)Si(OC₂H₅)₃, 3-aminopropyltrimethoxysilane (C₃H₆NH₂)Si(OCH₃)₃), (3-(methylamino)propyl)trimethoxysilane (C₃H₆NHCH₃)Si(OCH₃)₃, 3-carboxypropyltriethoxysilane (C₃H₆CO₂H)Si(OC₂H₅)₃, 3-carboxypropyltrimethoxysilane (Si(C₃H₆CO₂H) (OCH₃)₃), tetraethoxysilane and mixtures thereof, with 4 (dimethylamino) cinnamaldehyde in the solvent, said solvent comprising water, followed by addition of polystyrenesulfonic acid.

The addition of polystyrenesulfonic acid is preferably performed dropwise, as dissolution of the acid in the mixture is exothermic. Alternatively, the addition of the polystyrenesulfonic acid solution can be performed by adding to the mixture of organosilyl precursor and 4-(dimethylamino) cinnamaldehyde in solvent, this mixture being maintained at a temperature below 10° C.

According to a preferred embodiment, step a) of the process according to the invention is characterized in that the mole ratio of organosilyl precursor to solvent ranges from 1/20 to ½, preferably from 1/18 to ⅓, even more preferentially from 1/16 to ¼.

Advantageously, step a) of the process according to the invention is performed at room temperature, the term “room temperature” denoting a temperature of about 20-22° C.

According to a preferred embodiment, the mixture obtained at the end of step a) is kept stirring prior to gelling step b). Preferably, this stirring is performed at room temperature for a period ranging from 2 to 48 hours, preferably 24 hours.

The gelling step b) may be performed after the mixture obtained at the end of step a) has been poured into a mold. When a mold has thus been used, the process according to the invention may then include, after drying step c), a step for demolding the nanoporous sensor.

The gelling step b) may advantageously be performed at a relative humidity of 100%, at a temperature of between 2° and 25° C., preferably 22° C. This step b) preferably has a duration of 1 to 15 days, preferably 1 to 5 days, even more preferentially 2 days.

Drying step c) may advantageously be performed in a closed chamber, for example a desiccator, where humidity and temperature are preferably controlled.

Drying step c) is advantageously performed by flushing the closed chamber with a stream of moist, inert gas, preferably argon. The relative humidity of the applied gas stream can advantageously be controlled, being initially 100%, and then reduced in stages to about 25%. The humidity in the closed chamber may thus be reduced during the drying step to reach 25-28% relative humidity.

This step c) preferably lasts from 15 to 60 days, preferably from 20 to 40 days.

After drying, the nanoporous sensor obtained is preferentially stored protected from light, at a temperature ranging from 2 to 10° C., preferably from 4 to 8° C., even more preferentially 6° C.

The nanoporous sensor according to the invention and obtainable via the process of the invention advantageously has a parallelepiped shape. It advantageously has dimensions of the order of a millimeter, for example a height of 7.5 to 10.3 mm, a width of 4.8 to 6.4 mm and a thickness of 1 to 1.3 mm.

Another subject of the present invention is a process for detecting in a gas sample to be analyzed at least one amine-type compound, said compound being chosen from hydrazine, ethanolamine, ammonia and morpholine, using a nanoporous sensor according to the invention, said process comprising the steps of:
a. placing said gas sample to be analyzed in contact with said nanoporous sensor,
b. detecting on said nanoporous sensor said amine-type compound(s) in the gas sample to be analyzed.

This process advantageously allows selective measurement of an amine-type compound, in particular hydrazine, in a direct manner, in a concentration range from 1 to 100 ppb in dry to very humid air and in the presence of interferents whose total concentration may be at most 200 times higher than that of the amine to be detected, in particular hydrazine.

For the purposes of the present invention, the term “dry air” means air with a moisture content less than or equal to 25%.

Thus, the process according to the invention allows measurements to be performed on a gas sample with a relative humidity ranging between 25% and 100%, preferably from 30% to 100%.

The process according to the invention thus offers real advantages relative to the prior art processes which do not allow this measurement to be performed directly, whatever the moisture content, over this concentration range and in the presence of interferents.

In addition to detection, the process according to the invention may also allow determination in a gas sample to be analyzed of the concentration of at least one amine-type compound, said compound being chosen from hydrazine, ethanolamine, ammonia and morpholine. Thus, according to a particular embodiment, the present invention relates to a process for detecting and quantifying in a gas sample to be analyzed at least one amine-type compound, said compound being chosen from hydrazine, ethanolamine, ammonia and morpholine, using a nanoporous sensor according to the invention, said process comprising the steps of:
a. placing said gas sample to be analyzed in contact with said nanoporous sensor,
b. detecting and quantifying on said nanoporous sensor said amine-type compound(s) in the gas sample to be analyzed.

Step b) of the process according to the invention may notably comprise a step of measuring the absorbance of the nanoporous sensor as a function of time.

The analytical method which allows the detection and determination of the contents of hydrazine and interferents bearing a primary or secondary amine function by absorbance measurements as a function of time involves knowledge of the absorption spectra of the 4-(dimethylamino) cinnamaldehyde (DMACA)/polystyrenesulfonic acid (PSS) mixture in the presence of the amine-type compounds targeted for different concentrations of said amine compounds.

From these absorption spectra, calibration curves representing the temporal evolution of absorbance as a function of concentration for each of said amine-type compounds can be established.

Thus, when the sensor according to the invention is placed in the presence of a gas sample to be analyzed, the absorbance measurement result obtained as a function of time can be correlated with the calibration curves previously established, leading to the determination of the presence or absence of an amine-type compound and optionally to the determination of its concentration.

Detection and, optionally, quantification of hydrazine, ethanolamine, ammonia or morpholine by absorbance measurements is made possible by virtue of the reaction taking place between these compounds and 4-(dimethylamino) cinnamaldehyde (DMACA) in the presence of polystyrenesulfonic acid (PSS), which acts as a catalyst and results in the formation of complexes which have absorption spectra that are detectable in the UV-visible range, for example using a spectrophotometer.

Thus, the reaction between N₂H₄ and DMACA catalyzed in the presence of polystyrenesulfonic acid gives rise to the formation of the addition complexes IDMACA-N₂H₄ and 2DMACA-N₂H₄ according to the equations represented in FIG. 1.

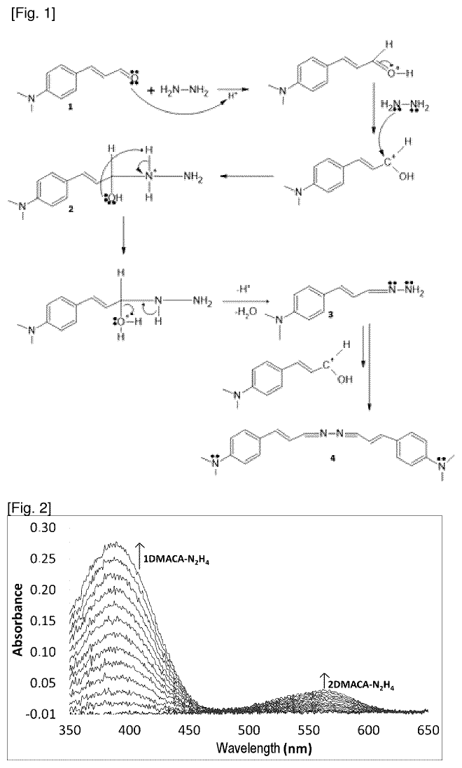

The formation of these two complexes 1DMACA-N₂H₄ and 2DMACA-N₂H₄ may be visualized by absorbance peaks at 388 and 558 nm respectively ([FIG. 2]).

In the context of the present invention, the peak at 388 nm is used to detect and quantify hydrazine, as it is the more sensitive of the two peaks formed. Specifically, the 2DMACA-N₂H₄ complex is formed more slowly and in smaller amounts than the DMACA-N₂H₄ complex for steric and kinetic reasons.

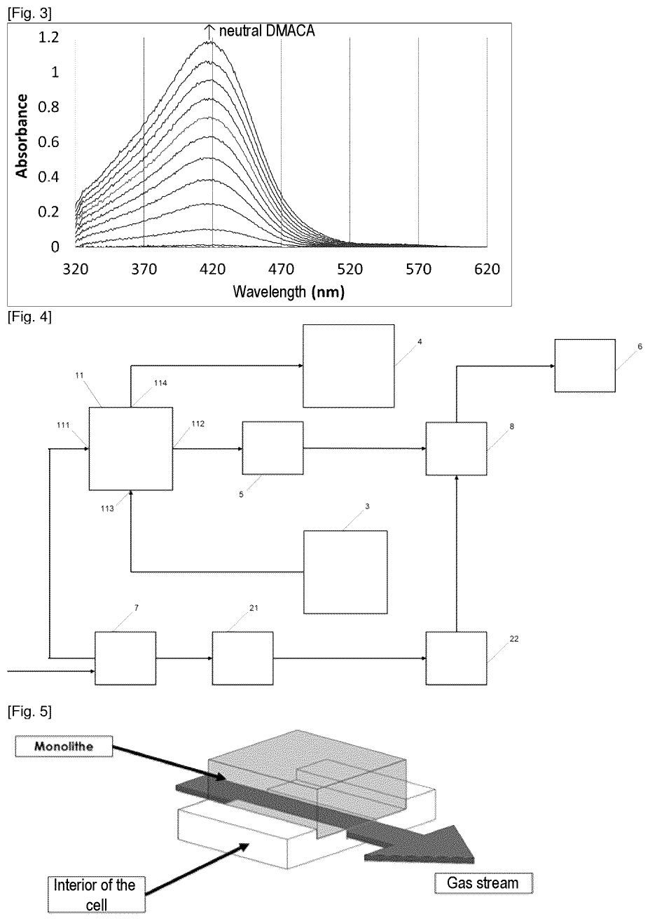

In the case of ammonia, DMACA (H⁺) deprotonates in the presence of a strong base such as NH₃, to form neutral DMACA, absorbing intensely at 420 nm ([FIG. 3]). Detection and measurement of the NH₃ concentration may thus be performed by measuring the deprotonation rate (corresponding to the ratio of absorbance variation to time) as a function of the NH₃ concentration in the gas mixture ([FIG. 7]).

Another subject of the present invention is the use of a nanoporous sensor according to the invention for the detection, or quantification, or detection and quantification of at least one amine-type compound, said compound being chosen from hydrazine, ethanolamine, ammonia and morpholine.

The nanoporous sensor according to the invention can be used in either dynamic or static mode. Thus, the nanoporous sensor may be placed in a gas stream to be analyzed and circulated by a fluidic circuit, or alternatively the sensor may be placed in a chamber comprising the gas sample to be analyzed.

Another subject of the present invention is a device (1) for detecting at least one amine-type compound, said compound being chosen from hydrazine, ethanolamine, ammonia and morpholine, in a gas sample to be analyzed, said device comprising a cell (11) containing a nanoporous sensor according to the invention, said cell comprising:
a gas inlet (111);
a gas outlet (112);
an optical input (113);
an optical output (114).

Advantageously, the device according to the invention includes a fluidic circuit suitable for circulating the gas sample to be analyzed through the cell (11), generating a gas stream from the gas inlet (111) to the nanoporous sensor and then to the gas outlet (112).

According to a preferred embodiment, the fluidic circuit according to the invention comprises a flow regulator (2) suitable for regulating the flow rate of the gas stream. The flow rate regulator (2) may be composed of a shut-off valve (21) and a control needle valve (22).

Advantageously, the device according to the invention also comprises a light source (3) and a spectrophotometer (4), in which the optical input (113) of the cell is connected to the light source (3) and the optical output (114) of the cell is connected to the spectrophotometer (4). The light source (3) and the spectrophotometer (4) may thus constitute the optical analysis part of the device.

According to a preferred embodiment, the fluidic circuit of the device according to the invention also comprises a temperature sensor suitable for measuring the temperature of the gas stream.

Advantageously, the device according to the invention also comprises a humidity sensor suitable for measuring the humidity of the gas stream.

Advantageously, the device according to the invention also comprises a flow meter (5) suitable for measuring the gas stream flow rate.

The fluidic circuit may thus take account of:
the gas mixture flow required to expose the sensors, which may possibly be varied, for example between 50 and 600 mL/min depending on the sensor,
measuring the temperature and relative humidity of the gas mixture,
a measurement circuit purge mode.

The device according to the invention may thus operate in the following manner: the air or gas sample to be analyzed can be sucked in using a pump (6), which may be miniature, and initially passed through an inlet manifold (7), where the temperature and humidity of the gas sample are measured. The manifold (7) can then distribute the gas sample to two outlets. The pump may notably have a flow rate ranging from 50 to 1100 ml/min.

The first outlet may move air toward the cell (11) containing the nanoporous sensor. The cell may be placed upstream of a flow meter (5) connected to an output manifold (8), which is itself connected to the pump (6). The flow meter allows the gas stream velocity in the exposure cell to be set.

The second outlet of the inlet manifold (7) may be connected to the outlet manifold (8) via a shut-off valve (21) and a control needle valve (22). This assembly then constitutes a leakage circuit. This configuration allows the exposure flow rate of the sensors to be varied (if different types of sensor are used), as the pump operates continuously at a fixed flow rate, for example 1.1 L/min. The shut-off valve allows the leakage circuit to be shut off, in order to purge the measuring circuit if necessary.

The optical analysis part includes a light source (3) which can probe the sensor and a spectrophotometer (4), the latter possibly being miniature, which collects the light transmitted by the sensor and transforms it into electrical current. The spectrophotometer (4) can operate at wavelengths ranging from UV through visible to IR, the wavelength being chosen according to the measurement to be performed.

The light source (3) may consist of two LEDs linked by optical fibers, or of a miniature lamp. The light source may be a UV or visible or UV-visible source.

For the detection of hydrazine at 388 nm, NH₃ at 420 nm, ethanolamine at 474 nm or morpholine at 490 nm, the combination of a UV LED and a visible LED affords the 380-800 nm wavelength range. Other LED choices are also possible, depending on the optical response of the sensor. The light is conveyed to the exposure cell, for example, using an optical fiber. At the output of the exposure cell, the transmitted light is conveyed to the miniature spectrophotometer (4), which has a wide response range, for example from 337.5 to 822 nm. An example of a device according to the invention is represented in [FIG. 4].

According to a preferred embodiment, the device according to the invention is characterized in that the cell comprises a nanoporous sensor support and a parallelepipedic, preferably cubic, case comprising four side faces and a front face forming at its center a housing for receiving the nanoporous sensor support, two opposite side faces presenting the gas inlet and outlet, two opposite side faces presenting the optical input and output, the front face having an aperture for inserting the nanoporous sensor support into its housing (FIG. 6).

The cell of the device according to the invention may thus include two elements.

The first element is its body, which may consist of a square block (FIG. 6) of brass reinforced, for example, by an internal layer of stainless steel, and which may notably be hollowed out in its center and perforated through on all four sides. Two perpendicular tunnels may thus be created to serve as passages for the gas stream and the probe light. This block may thus be equipped with an optical input and output, and also a gas inlet and outlet.

The second element may be a removable part, made of stainless steel for example (FIG. 6), which may also be perforated on all four sides to allow the gas stream and light to pass through. Sealing of the two tunnels between the external block and the removable part may be achieved using O-rings. The removable part may contain at its center the nanoporous sensor, which may be, for example, a transparent monolithic block with maximum dimensions of about ten millimeters, for example 11×6.5×2 mm. The optical input and output may each include a lens for collimating the analysis light beam. The optical input may be connected via an optical fiber to a UV or Visible light source or a combination of both UV-Visible, and the optical output may be connected to the UV or Visible or UV-Visible spectrophotometer required for analysis and compatible with the lamp, via a second optical fiber.

An example of a cell of the device according to the invention is represented in [FIG. 6].

The sensor may be exposed to the gas stream over its entire length and on both sides. The probe light beam, coming from the light source and carried by an optical fiber, arrives perpendicular to the gas stream and is focused on the sensor at the fiber outlet. The probed surface may be a 5.7 mm² circle (2.7 mm diameter). The optical path of analysis, which may range from 1 to 2 mm, corresponds to the thickness of the inserted nanoporous sensor.

Depending on the sensor's geometry, for example a disk or a parallelepiped, the removable part may be designed to position the sensor correctly and expose it to the gas stream over its largest surface area.

According to a preferred embodiment, the sensor is positioned between two half-cylinders, hollowed out to allow the gas stream to pass above and below the sensor ([FIG. 5] and [FIG. 6]). The sensor is positioned at the intersection of a vertical hole through which the probe light can pass.

According to a preferred embodiment, the device according to the invention is characterized in that the nanoporous sensor support comprises two diametrically opposed passage orifices, optically connected to the optical input and output.

Advantageously, the device according to the invention also comprises a computer and a battery to provide electrical power to said device and thus make it energy self-sufficient.

The device according to the invention may also comprise a screen allowing the user to interact with the system's elements by computer.

The device according to the invention may also comprise a power supply and control board.

The components of the device, composed of the fluidic circuit and the optical analysis part, may thus be positioned in a compartment of a portable element. This portable element may then include a second compartment containing a computer suitable for controlling the measurements, and a third compartment including a power supply for operating the device as a whole.

Thus, according to a particular embodiment, the device according to the invention may be integrated into a case comprising several levels, notably three levels. The computer may, for example, be located on the upper level of the case, the device according to the invention on the middle level and the power supply on the lower level.

According to a preferred embodiment, the device according to the invention is characterized in that it is portable.

Advantageously, the device according to the invention allows quantification of said amine-type compound(s) in the gas sample to be analyzed.

### Examples

### Example 1: Synthesis of Nanoporous Sensors According to the Invention

### Reagents Used:

Tetramethyl orthosilicate (TMOS), purity 99%, CAS: 681-84-5, molar mass=152.22 g·mol⁻¹ and density d=1.023 g·cm⁻³
4-(Dimethylamino) cinnamaldehyde (DMACA), purity ≥98%, CAS: 6203-18-5, molar mass=175.23 g·mol⁻¹
Polystyrenesulfonic acid (PSS), 30% aqueous solution, CAS: 28210-41-5, molar mass=75 000 g·mol⁻¹ and density d=1.1 g·cm⁻³
para-Toluenesulfonic acid (C₇H₇—SO₃H), purity ≥98%, CAS: 6192-52-5, molar mass=190.22 g·mol⁻¹
Ultra-pure deionized water.

### Example 1.1: Hy1 Sensor

In a 1 L bottle, 14.2 mg of DMACA, 40.969 mL of H₂O and 84.598 mL of TMOS are mixed. The solution is kept stirring at room temperature and 0.449 mL of 30% PSS is added dropwise, as the dissolution of PSS in the mixture is exothermic. The mole ratio of silylated precursor to water in the mixture is TMOS/H₂O=1/4. The respective final concentrations of DMACA and PSS are 6.25×10⁻⁴ M and 1.18 g·L⁻¹, i.e. [H⁺]˜6.25×10⁻³ M.

The sol is kept stirring for 24 h at room temperature and then poured into a polypropylene mold containing 350 wells of 0.3 cm³ volume. The mold is placed in a 10 L desiccator maintained at 100% relative humidity until the sol gels. This step takes 2 days at 22° C. for Hy1. Drying of the gels is then performed by flushing the desiccator with a stream of moist Ar at 300 mL·min⁻¹. The relative humidity of the Ar stream, initially 100%, is reduced in stages to 80%, 50% and then 0%. Drying takes about 1 month at 22° C., during which the humidity in the desiccator reduces to 25-28% relative humidity. The mold is then removed from the desiccator. After removing from the mold, parallelepiped sensors with dimensions of 9.6(H)*6.0(L)*1.26 (thickness) mm are obtained for an initial solution of 0.3 mL. The final volume of the sensors obtained has shrunk by a factor of 4.2. The sensors are stored in a cool place at 6° C., protected from light.

### Example 1.2: Hy2 Sensor

Same procedure as Hy1 with 14.2 mg of DMACA, 84.598 mL of TMOS, 40.969 mL of deionized H₂O and 0.673 mL of 30% PSS. The mole ratio of silylated precursor to water in the mixture is TMOS/H₂O=1/4. The respective final concentrations of DMACA and PSS are 6.25×10⁻⁴ M and 1.77 g·L⁻¹, i.e. [H⁺]˜9.38×10⁻³ M. The gelling time of the sensors in the desiccator maintained at RH=100% is 2 days at 22° C. Drying in stages from RH=80%, 50% to 0% takes about 40 days at 22° C., during which the humidity in the desiccator reduces to 25-28% relative humidity. After removing from the mold, sensors with dimensions of 9.7(H)*6.1(L)*1.26 (thickness) mm and a shrinkage factor of 4 are obtained. The sensors are stored in a cool place at 6° C., protected from light.

### Example 1.3: Hy3 Sensor

Same procedure as Hy1 with 28.5 mg of DMACA, 84.598 mL of TMOS, 40.969 mL of deionized H₂O and 0.898 mL of 30% PSS. The mole ratio of silylated precursor to water in the mixture is TMOS/H₂O=1/4. The respective final concentrations of DMACA and PSS are 1.25×10⁻³ M and 2.34 g·L⁻¹, i.e. [H⁺]˜1.25×10⁻² M. The gelling time of the sensors in the desiccator maintained at RH=100% is 2 days at 22° C. Drying in stages from RH=80%, 50% to 0% takes about 1 month at 22° C., during which the humidity in the desiccator reduces to 25-28% relative humidity. After removing from the mold, sensors with dimensions of 9.5(H)*6.1(L)*1.24 (thickness) mm and a shrinkage factor of 4.2 are obtained. The sensors are stored in a cool place at 6° C., protected from light.

### Example 1.4: Hy4 Sensor

Same procedure as Hy1 with 113 mg of DMACA, 81.496 mL of H₂O and 43.91 mL of TMOS and 3.563 mL of 30% PSS. The mole ratio of silylated precursor to water in the mixture is TMOS/H₂O=1/16. The respective final concentrations of DMACA and PSS are 5×10⁻³ M and 9.2 g·L⁻¹, i.e. [H⁺]˜5×10⁻² M. Gelation of the sensors in the desiccator maintained at RH=100% takes place after 5 days at 22° C. Drying in stages from RH=80%, 50% to 0% takes about 1.5 months at 22° C., during which the humidity in the desiccator reduces to 25-28% relative humidity. After removing from the mold, sensors with dimensions of 8.37(H)*4.94(L)*1.03 (thickness) mm and a shrinkage factor of 7 are obtained. The sensors are stored in a cool place at 6° C., protected from light.

### Example 1.5: Hy5 Sensor

Same procedure as Hy1 with 87.9 mg of DMACA, 33.777 mL of TMOS, 13.587 mL of H₂O and 2.77 mL of 30% PSS. The mole ratio of silylated precursor to water in the mixture is TMOS/H₂O=1/4. The respective final concentrations of DMACA and PSS are 1×10⁻² M and 18.4 g·L⁻¹, i.e. [H⁺]˜1×10⁻¹ M. The gelling time of the sensors in the desiccator maintained at RH=100% is 5 days at 22° C. Drying in stages from RH=80%, 50% to 0% takes about 1 month at 22° C., during which the humidity in the desiccator reduces to 25-28% relative humidity. After removing from the mold, sensors with dimensions of 10.2(H)*6.4(L)*1.3 (thickness) mm and a shrinkage factor of 3.5 are obtained. The sensors are stored in a cool place at 6° C., protected from light.

### Example 1.6: Hy6 Sensor

Same procedure as Hy1, with 226 mg of DMACA, 43.91 mL of TMOS, 77.933 mL of water and 7.126 mL of PSS. The mole ratio of silylated precursor to water in the mixture is TMOS/H₂O=1/16. The final concentrations of DMACA and PSS are 1×10⁻² M and 18.4 g·L⁻¹, i.e. [H⁺]˜1×10⁻¹ M. The gelling time of the sensors in the desiccator maintained at RH=100% is 3 days at 22° C. Drying in stages from RH=80%, 50% to 0% takes about 49 days at 22° C., during which the humidity in the desiccator reduces to 25-28% relative humidity. After removing from the mold, sensors with dimensions of 8.4(H)*5.0(L)*1.05 (thickness) mm sensors and a shrinkage factor of 6.7 are obtained.

### Example 1.7: Hy7 Sensor

56.5 mg of DMACA, 21.258 mL of H₂O and 10.974 mL of TMOS are mixed in a 1 L bottle. The solution is kept stirring at room temperature while slowly adding 613 mg of para-toluenesulfonic acid, C₇H₇—SO₃H. When the acid is added to this mixture, the mixture releases heat. The mole ratio of silylated precursor to water is TMOS/H₂O=1/16. The respective final concentrations of DMACA and para-toluenesulfonic acid are 1×10⁻² M and 1×10⁻¹ M.

The sol is kept stirring for 3 h at room temperature and then poured into a polypropylene mold. The mold is placed in a 10 L desiccator maintained at 100% relative humidity until the sol gels. This step takes 5 days at 22° C. Drying is performed by flushing the desiccator with an Ar stream of 300 mL·min⁻¹, while gradually reducing the relative humidity in the desiccator from 100% to 80%, 50% and then 0% RH. Drying takes about 1 month at 22° C., during which the humidity in the desiccator reduces to 25-28% RH. The mold is removed from the desiccator. After removing from the mold, parallelepiped-shaped sensors with dimensions of 7.57(H)*4.8(L)*1.0 (thickness) mm and a shrinkage factor of 8.2 are obtained. The sensors are stored in a cool place at 6° C., protected from light.

### Example 1.8: Hy8 Sensor

Same procedure as Hy1 with 1.139 g of DMACA, 84.598 mL of TMOS, 5.054 and 35.916 mL of 30% PSS. The respective final concentrations of DMACA and PSS are 5×10⁻² M and 95.2 g·L⁻¹, i.e. [H⁺]˜5×10⁻¹ M. The gelling time of the sensors in the desiccator maintained at RH=100% is 4 days at 22° C. Drying in stages from RH=80%, 50% to 0% takes about 1 month at 22° C., during which the humidity in the desiccator reduces to 25-28% relative humidity. After removing from the mold, sensors with dimensions of 11.2(H)*6.4(L)*1.2 (thickness) mm and a shrinkage factor of 3.4 are obtained.

### Example 1.9: Hy9 Sensor

Same procedure as Hy1 with 596.5 mg of DMACA, 84.598 mL of TMOS, 23.012 mL of water and 17.958 mL of 30% PSS. The mole ratio of silylated precursor to water is TMOS/H₂O=1/4. The respective final concentrations of DMACA and PSS are 2.5×10⁻² M and 47.6 g·L⁻¹, i.e. [H⁺]˜2.5×10⁻¹ M. The gelling time of the sensors in the desiccator maintained at RH=100% is 2 days at 22° C. Drying in stages from RH=80%, 50% to 0% takes about 1 month at 22° C., during which the humidity in the desiccator reduces to 25-28% relative humidity. After removing from the mold, sensors with dimensions of 10.24(H)*6.5(L)*1.25 (thickness) mm and a shrinkage factor of 3.6 are obtained.

The porosity properties of the nanoporous sensors, such as specific surface area for adsorption, pore volume or the size distributions of the micropores and mesopores, were determined by establishing adsorption-desorption isotherms for N₂ at the temperature of liquid N₂. The table below collates these data.

TABLE 1

| | | | | | | Porosity properties | | | |
|---|---|---|---|---|---|---|---|---|---|
| | | Concentration of reagents in | | | | | | % of | |
| | | the sol (mol · L)⁻¹ | | | | | | μpore | % of |
| Sensor | Formulation (TMOS/H₂O) | [DMACA] | [monomer PSS] = [H]⁺ | $\frac{[ {H}^{+} ]}{[ DMACA ]}$ | $\frac{{V}_{sol}}{{V}_{solid}}$ | S_{DFT}/ m² · g⁻¹ | Vₚₒᵣₑ/ cm³ · g⁻¹ | Size in Å | mesopore Size in Å |
| Hy1 | 1/4 in moles | 6.25 × 10⁻⁴ | 6.25 × 10⁻³ | 10 | 4.2 | 1794 | 0.33 | 100% | 0% |
| | | | | | | | | 5 < d < 19 | |
| Hy2 | 1/4 in moles | 6.25 × 10⁻⁴ | 9.38 × 10⁻³ | 15 | 4.0 | 1929 | 0.44 | 95% | 5% |
| | | | | | | | | 5 < d < 20 | 20 < d < 88 |
| Hy3 | 1/4 in moles | 1.25 × 10⁻³ | 1.25 × 10⁻² | 10 | 4.2 | 1878 | 0.4 | 97% | 3% |
| | | | | | | | | 5 < d < 20 | 20 < d < 65 |
| Hy4 | 1/16 in moles | 5 × 10⁻³ | 5 × 10⁻² | 10 | 7.0 | 1478 | 0.51 | 82% | 18% |
| | | | | | | | | 5 < d < 12 | 20 < d < 131 |
| Hy5 | 1/4 in moles | 1 × 10⁻² | 1 × 10⁻¹ | 10 | 3.5 | 1593 | 0.42 | 90% | 10% |
| | | | | | | | | 5 < d < 20 | 20 < d < 102 |
| Hy6 | 1/16 in moles | 1 × 10⁻² | 1 × 10⁻¹ | 10 | 6.7 | 1414 | 0.42 | 86% | 14% |
| | | | | | | | | 5 < d < 20 | 20 < d < 88 |
| Hy7 | 1/16 in moles | 1 × 10⁻² | 1 × 10⁻¹ | 10 | 8.2 | 853 | 0.24 | 88% | 12% |
| | | | (C₇H₇— | | | | | 6 < d < 20 | 20 < d < 61 |
| | | | SO₃H) | | | | | | |
| Hy8 | 1/4 in moles | 5 × 10⁻² | 5 × 10⁻¹ | 10 | 3.4 | 1778 | 0.52 | 89% | 11% |
| | | | | | | | | 8 < d < 13 | 20 < d < 131 |
| Hy9 | 1/4 in moles | 2.5 × 10⁻² | 2.5 × 10⁻¹ | 10 | 3.6 | 1608 | 0.49 | 87% | 13% |
| | | | | | | | | 5 < d < 20 | 20 < d < 107 |

The percentages of micropores and mesopores given here correspond to the distribution of adsorption surface area as a function of pore diameter. This percentage would be different considering the pore volume distribution as a function of pore diameter.

### Example 2: Hy1 Sensor Response to NH₃

The Hy1 sensor was exposed to a humid gas mixture (relative humidity RH=50%) containing 5 ppm of NH₃. NH₃ does not react with DMACA (H⁺) but induces deprotonation of DMACA (H⁺) to form neutral DMACA. In the porous material, neutral DMACA exhibits a broad absorption band in the UV-near visible range, with the maximum centered at 420 nm (FIG. 7). The Hy1 sensor calibration curve corresponding to the rate of formation of neutral DMACA as a function of NH₃ concentration is shown in FIG. 7.

### Example 3: Hy3 Sensor Response to N₂H₄. Establishing a Calibration Curve for N₂H₄ Gas

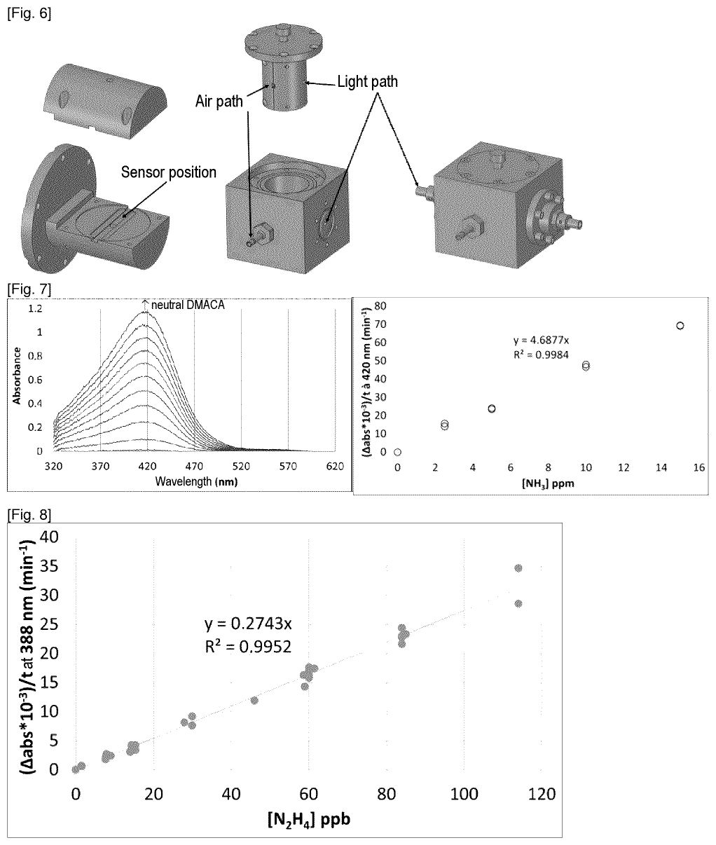

Hy3 sensors are exposed to different concentrations of N₂H₄ over a wide concentration range from 1 to 114 ppb. The relative humidity of the gas mixtures was kept fixed at 50%. For each exposure to a given N₂H₄ content, the rate of formation of the IDMACA-N₂H₄ complex at 388 nm was deduced. By plotting the rate of formation of the IDMACA-N₂H₄ complex as a function of N₂H₄ concentration, a calibration curve for the detection of N₂H₄ at 388 nm is obtained. An example of an N₂H₄ calibration curve established for the Hy3 sensor is shown in FIG. 8.

FIG. 8 shows the calibration curve for N₂H₄ produced by exposing Hy3 sensors with storage times ranging from five months to fifteen months. A linear variation in IDMACA-N₂H₄ complex formation rates is obtained as a function of N₂H₄ concentration. The detection limit is 1 ppb for a probed gas volume of 12 L (60 minutes, 200 mL·min⁻¹ and ΔAbs=0.02). The exposure time may be reduced by increasing the flow rate of the gas mixture to be analyzed.

### Example 4: Response of Hy3 Sensors to N₂H₄, at Different Relative Humidities

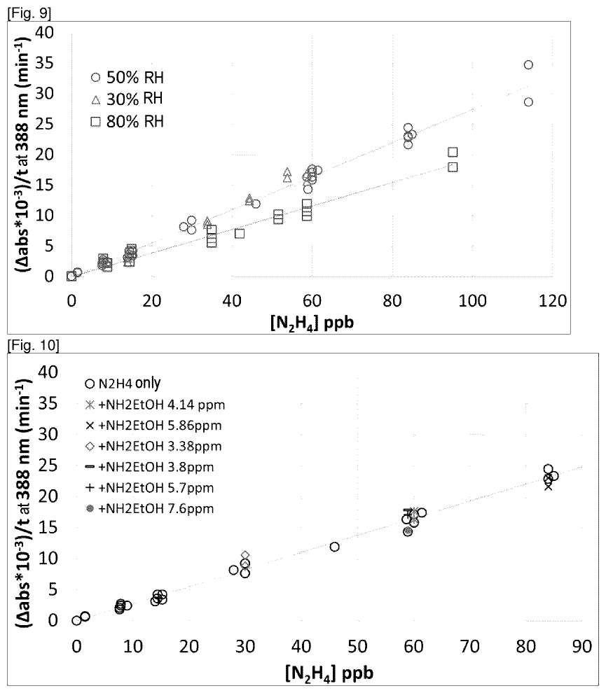

The effect of the relative humidity of gas mixtures on the N₂H₄ calibration curve of the Hy3 sensor is investigated ([FIG. 9]). To this end, the Hy3 sensors are exposed to different concentrations of N₂H₄ in gas mixtures at 30%, 50% and 80% RH.

### Example 5: Hy3 Sensor Response to N₂H₄ in the Presence of a Potential Interferent, NH₂EtOH

The response of the Hy3 sensor to N₂H₄ in the presence of NH₂EtOH is studied ([FIG. 10]). To this end, the Hy3 sensors are exposed to gas mixtures containing N₂H₄+NH₂EtOH.

### Example 6: Hy3 Sensor Response to N₂H₄ in the Presence of a Potential Interferent, Morpholine

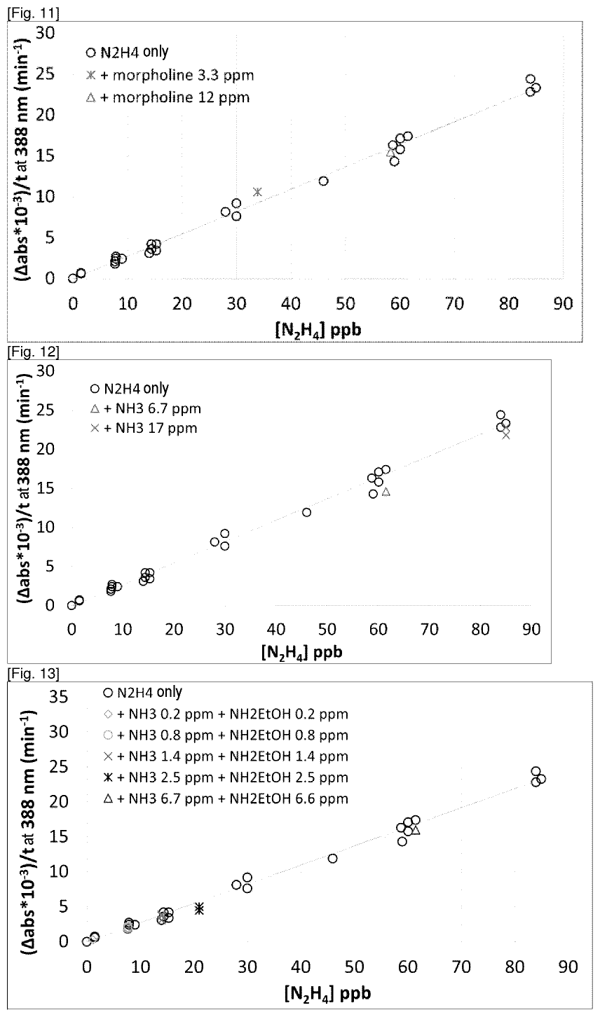

The response of the Hy3 sensor to N₂H₄ in the presence of morpholine is studied ([FIG. 11]). To this end, the Hy3 sensors are exposed to gas mixtures containing N₂H₄+morpholine.

### Example 7: Hy3 Sensor Response to N₂H₄ in the Presence of a Potential Interferent, NH₃

The response of the Hy3 sensor to N₂H₄ in the presence of NH₃ is studied ([FIG. 12]). To this end, the Hy3 sensors are exposed to gas mixtures containing N₂H₄+NH₃.

### Example 8: Hy3 Sensor Response to N₂H₄ in the Presence of the Two Potential Interferents, NH₃ and NH₂EtOH

The response of the Hy3 sensor to N₂H₄ in the presence of the two potential interferents, NH₃ and NH₂EtOH, is studied ([FIG. 13]). To this end, the Hy3 sensors are exposed to gas mixtures containing N₂H₄+NH₃+NH₂EtOH.

### Example 9: Hy3 Sensor Response to N₂H₄ in the Presence of Two Potential Interferents, NH₃ and Morpholine

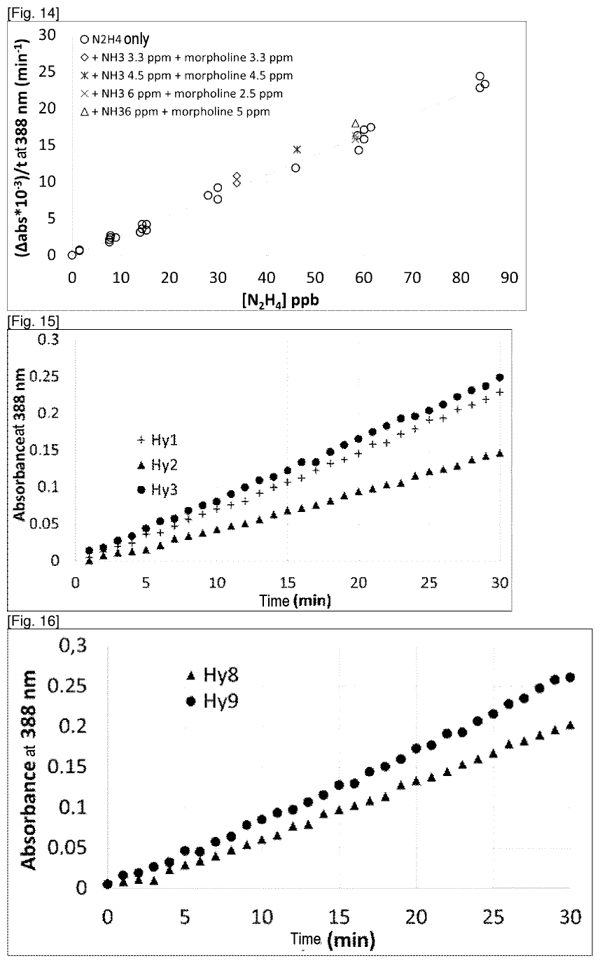

The response of the Hy3 sensor to N₂H₄ in the presence of the interferents, NH₃ and morpholine, is studied ([FIG. 14]). To this end, the Hy3 sensors are exposed to gas mixtures containing N₂H₄+NH₃+morpholine.

### Example 10: Comparison of the Responses of Hy1, Hy2 and Hy3 Sensors to 30 Ppb of N₂H₄

The responses of the Hy1, Hy2 and Hy3 sensors to N₂H₄ are studied ([FIG. 15]). To this end, the Hy1, Hy2 and Hy3 sensors are exposed to 30 ppb of N₂H₄.

### Example 11: Comparison of Hy8 and Hy9 Sensors at 40 Ppb of N₂H₄

The responses of the Hy8 and Hy9 sensors to N₂H₄ are studied ([FIG. 16]). To this end, the Hy8 and Hy9 sensors are exposed to 40 ppb of N₂H₄.

### Example 12: Comparison of Sensors Hy4, Hy5, Hy6 and Hy7 at 25 Ppb of N₂H₄

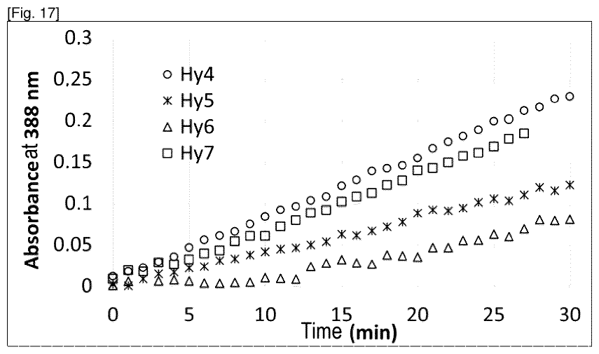

The responses of Hy4, Hy5, Hy6 and Hy7 sensors to N₂H₄ are studied ([FIG. 17]). To this end, the Hy4, Hy5, Hy6 and Hy7 sensors are exposed to 25 ppb of N₂H₄.

### LIST OF DOCUMENTS CITED

[¹] Toxicology data sheet No. 21 INRS http://www.inrs.fr/publications/bdd/fichetox/fiche.html?refINRS=FICHETOX_21
[²] Air quality. Workplace air. Sampling and analyzing organic vapors. Sampling by pumping through a solvent adsorption and desorption tube. Standard NF X 43-267. La Plaine Saint Denis: AFNOR; 2004.
[³] Métropole Hydrazine M-7 data sheet, http://www.inrs.fr/publications/bdd/metropol/fiche.html?refINRS=METROPOL_7
[⁴] Hydrazine. Method 108 In: Sampling and Analytical methods. OSHA, 1997 (https://www.osha.gov/dts/sltc/methods/organic/org108/org108.html)
[⁵] B. J. Meneghelli, A review of hydrazine sensors: The state of the art, ASRC Aerospace Corp, Cocoa Beach, FL, United States, 2004.
[⁶] K. P. Brenner, S. L. Rose-Pehrsson, Performance Evaluation of a Colorimetric Hydrazine Dosimeter, 1994.
[⁷] https://dodtec.com/hydrazine-mmh-dose-estimator.html
[⁸] https://www.chemsee.com/commercial/toxic-gas/available-products/dosimeters/hyd-009-dosimeter-for-hydrazine/[9]
[⁹] Rose-Pehrsson et al., patent U.S. Ser. No. 00/571,9061 A
[¹⁰] http://catalog.gasdetection.com/item/search-by-gas-type-hydrazine-s-/nalyzer-4000-series-with-digital-display-hydrazine/4180-100b
[¹¹] https://www.raefrance.fr/produit/detecteur-cov-capteur-pid-ppbrae-3000/
[¹²] https://www.cbrnetechindex.com/p/3525/Smiths-Detection-Inc/Sabre-4000
[¹³] https://www.environics.fi/product/chempro100i/
[¹⁴] https://www.masatech.eu/portable-advanced-ion-mobility-spectrometer
[¹⁵] https://www.smithsdetection.com/products/lcd-3-3/

1. A nanoporous sensor composed of a silicate sol-gel matrix containing a reagents composition, said reagents composition comprising a mixture of 4-(dimethylamino) cinnamaldehyde and polystyrenesulfonic acid.

2. The nanoporous sensor according to claim 1, wherein the nanoporous sensor has a specific surface area for adsorption of 700 to 2500 m²·g⁻¹.

3. The nanoporous sensor according to claim 1, wherein the nanoporous sensor has a pore volume of 0.1 to 0.9 cm³·g⁻¹.

4. The nanoporous sensor according to claim 1, wherein the nanoporous sensor has a proportion of micropores of greater than 75% corresponding to the proportion of mesopores.

5. A process for preparing the nanoporous sensor according to claim 1, said process comprising the following steps:
a) synthesis of a sol from an organosilyl precursor, the synthesis being performed in a solvent, said solvent comprising water, in the presence of 4-(dimethylamino) cinnamaldehyde and polystyrenesulfonic acid;
b) molding of the sol obtained in step a), followed by gelation to obtain a gel; and
c) drying of the gel obtained in step b), followed by removing from the mold to obtain a nanoporous sensor.

6. The process according to claim 5, wherein the organosilyl precursor is chosen from tetramethoxysilane, methyltrimethoxysilane, ethyltrimethoxysilane, 3-aminopropyltriethoxysilane, 3-aminopropyltrimethoxysilane, (3-(methylamino)propyl)trimethoxysilane, 3-carboxypropyltriethoxysilane, 3-carboxypropyltrimethoxysilane, tetraethoxysilane and mixtures thereof.

7. A process for detecting in a gas sample to be analyzed at least one amine-type compound, said compound being chosen from hydrazine, ethanolamine, ammonia and morpholine, using the nanoporous sensor according to claim 1, said process comprising the steps of:
a) placing said gas sample to be analyzed in contact with said nanoporous sensor, and
b) detecting on said nanoporous sensor said amine-type compound(s) in the gas sample to be analyzed.

8. The process according to claim 7, wherein step b) further comprises quantifying on said nanoporous sensor of said amine-type compound(s) in the gas sample to be analyzed.

9. A method for detecting or quantifying or detecting and quantifying at least one amine-type compound, said compound being chosen from hydrazine, ethanolamine, ammonia and morpholine comprising contacting a sample with the nanoporous sensor of claim 1.

10. A device for detecting in a gas sample to be analyzed at least one amine-type compound, said compound being chosen from hydrazine, ethanolamine, ammonia and morpholine, said device comprising:
a cell enclosing the nanoporous sensor according to claim 1 and comprising:
a gas inlet;
a gas outlet;
an optical input; and
an optical output.
